(19) United States
(12) Patent Application Publication
(10) Pub. No.: US 2006/0120979 A1
(43) Pub. Date: Jun. 8, 2006

(54) Skin care composition comprising hydroquinone and a substantially anhydrous base

(76) Inventor: Joel Rubin, Smithtown, NY (US)

Correspondence Address: WIGGIN AND DANA LLP;ATTENTION: PATENT DOCKETING, ONE CENTURY TOWER, P.O. BOX 1832, NEW HAVEN, CT, 06508-1832, US

(21) Appl. No.: 11/290,966

(22) Filed: Nov. 30, 2005

Related U.S. Application Data
(60) Provisional application No. 60/632,365, filed on Dec. 2, 2004.

(51) Int. Cl. A61K 8/89; A61K 8/34

(52) U.S. Cl. 424/62; 424/70.12; 424/59

(57) ABSTRACT

The present invention is directed to an anhydrous topical skin care composition, comprising: hydroquinone and an anhydrous base comprising an anhydrous solvent and a high molecular weight silicone carrier. The composition is substantially free of water which imparts advantageous properties, including smooth feel and long shelf life, to the composition.

### CROSS-REFERENCE TO RELATED APPLICATIONS

This application claims priority to Provisional Patent Application No. 60/632,365 filed Dec. 2, 2004.

### BACKGROUND OF THE INVENTION

1. Field of the Invention

The present invention is directed to topical skin care composition, and more particularly to topical skin care composition that include hydroquinone in an anhydrous base.

2. Description of the Related Art

Hydroquinone (also known as 1,4-benzenediol, p-dihydroxybenzene, and hydroquinol) has been known in the skin care field for many years as a depigmenting or bleaching agent. Skin care products containing hydroquinone are commonly prescribed by dermatologists to lighten hyperpigmentation, such as age spots, freckles, or dark spots related to pregnancy or hormone therapy (melasma or chloasma), and give the skin a more event and attractive appearance. Hydroquinone is thought to work by inhibiting tyrosinase activity of the melanin cells in the basal layer of the epidermis.

Most skin treatment compositions that contain hydroquinone are manufactured in an aqueous (water) base, generally for cost reasons. However, this aqueous base results in several disadvantages for the final product. First, hydroquinone is not readily soluble in aqueous solutions. This insolubility can result in crystals of hydroquinone in the final product, which many consumers find irritating when applied to the skin. Second, hydroquinone will often degrade in an aqueous solution, which results in reduced effectiveness. To combat this degradation, reducing agents, such as sulfites, are added to the composition as a preservative. However, these preservatives are irritating to the skin and give off an unpleasant odor.

A variety of skin care compositions use hydroquinone as an ingredient, including the following:

U.S. Pat. No. 5,505,948 to Rapaport discloses a composition, method and kit for at-home chemical skin peeling. The method of the invention includes a skin bleaching step that utilizes hydroquinone.

U.S. Pat. No. 5,962,018 to Curtis et al. discloses methods of treating skin with organic acids in an anhydrous microsphere delivery system. A skin lightening agent, such as hydroquinone, may be included in the delivery system.

U.S. Pat. No. 5,993,787 to Sun et al. discloses an anhydrous topical preparation made from propylene carbonate, one or more short carbon chain alcohols and/or glycols, and either or both of glycerol and one or more therapeutically or cosmetically active ingredients. The preparation may include one or more of a plethora of additional ingredients, including hydroquinone among others.

U.S. Pat. No. 6,607,736 to Ohmori et al. discloses an endermic liniment comprising polyoxyethylene dicarboxylates. The disclosed liniment may include one or more of a plethora of additional ingredients, including hydroquinone among others.

U.S. Pat. No. 6,682,750 to Loffler et al. discloses a surfactant-free cosmetic, dermatological or pharmaceutical composition made from at least one of a series of defined crosslinked copolymers. Hydroquinone is disclosed as a suitable crosslinking agent.

U.S. Pat. No. 6,682,763 to Kuno et al. discloses skin care compositions based on herbal or botanical extracts. Hydroquinone is mentioned as a common skin bleaching agent.

What is needed in the art is a skin care composition that includes hydroquinone as an active ingredient, but does not contain crystals or require irritating or malodorous preservatives to maintain the effectiveness of the active ingredient. The present invention is believed to be an answer to that need.

### SUMMARY OF THE INVENTION

In one aspect, the present invention is directed to an anhydrous topical skin care composition, comprising: hydroquinone and an anhydrous base comprising an anhydrous solvent and a high molecular weight silicone carrier; wherein the composition is substantially free of water.

In another aspect, the present invention is directed to a method of making an anhydrous topical skin care composition, comprising the steps of: (a) combining hydroquinone and an anhydrous solvent to form an anhydrous solvent containing dissolved hydroquinone; (b) combining the anhydrous solvent containing dissolved hydroquinone with a high molecular weight silicone carrier to form the anhydrous topical skin care composition.

These and other aspects will become apparent upon reading the following detailed description of the invention.

### DETAILED DESCRIPTION OF THE INVENTION

It has now been surprisingly found that a topical skin care composition may be made that includes hydroquinone in a substantially anhydrous base made from an anhydrous solvent and a high molecular weight silicone carrier. The present inventor has discovered that the anhydrous base portion of the composition of the invention provides numerous advantages to a skin care composition that includes hydroquinone as an active ingredient. For example, use of an anhydrous base significantly reduces water-induced degradation of the hydroquinone moiety. This reduced degradation eliminates the need for addition of irritating and/or malodorous reducing agents such as sulfites. Elimination of water from the composition also increases its shelf-life. In addition, the anhydrous base prevents formation of crystalline hydroquinone that can result in an undesirable grainy, rough-feeling product. The composition of the present invention advantageously possesses a smooth, elegant feel on the skin.

As indicated above, the present invention is directed to an anhydrous topical skin care composition, comprising: hydroquinone; and an anhydrous based comprising an anhydrous solvent and a high molecular weight silicone carrier, wherein the composition is substantially free of water. Each of these components is discussed in more detail below.

The active ingredient in the present composition is hydroquinone (also known as 1,4-benzenediol, p-dihydroxybenzene, and hydroquinol). Although any grade of hydroquinone is suitable for use in the present invention, preferably the hydroquinone is “USP Grade” or better. Hydroquinone suitable for this application is available from the Eastman Chemical Company, Kingsport, Tenn.

Hydroquinone is known to be freely soluble in alcohols, and less soluble in water. The hydroquinone in the composition of the invention is preferably dissolved in the anhydrous base portion of the composition so that no crystals of solid hydroquinone are present. Lack of crystals of hydroquinone prevents a grainy consistency in the composition, and contributes to an elegant feel of the composition against the skin. As disclosed in more detail below, the hydroquinone is first dissolved in an anhydrous solvent, such as butylene glycol, and then mixed with the remaining components of the composition.

Preferably, the amount of hydroquinone present in the composition of the invention ranges from about 0.5 to about 10% by weight, more preferably from about 1 to about 7% by weight, and most preferably from about 2 to about 4% by weight, all based on the total weight of the composition.

The anhydrous base portion of the present composition comprises (1) an anhydrous solvent and (2) a high molecular weight silicone carrier. Preferably, the anhydrous solvent is selected from the group consisting of monohydric alcohols (such as isopropanol), dihydric alcohols (such as glycols), trihydric alcohols (such as glycerols), and the like. Pharmaceutical or cosmetic grade materials are preferably used.

A dihydric alcohol, such as a glycol, is one in which the two hydroxyl groups are bonded to different carbon atoms, and has the general formula (CH₂)ₙ(OH)₂. Dihydric alcohols that are useful in the composition of the present invention include glycols such as butylene glycol, propylene glycol, pentylene glycol, ethylene glycol, polyethylene glycol, hexylene glycol, ethoxydiglycol, methoxypolyethylene glycol such as those sold under the tradename CARBOWAX, as well as combinations of these. Trihydric alcohols that are useful in the composition of the invention include glycerol and dimethyl isosorbide. Any of the anhydrous solvents disclosed above may be implemented in the composition of the invention singly, or in combinations of two, three, four, or more.

In the composition of the present invention, the anhydrous solvent preferably comprises from about 1 to about 20% by weight, more preferably from about 4 to about 14% by weight, and most preferably from about 4 to about 8% by weight, all based on the total weight of said composition.

The high molecular weight silicone carrier is included in the composition of the invention to provide aesthetic benefits for the formulations. Examples of useful high molecular weight silicones are well known in the art and include, for example, polydiorganosiloxane-polyoxyalkylene copolymers (e.g., Shin Etsu KF 6017, Dow Corning 5200), cross-linked silicone elastomers (e.g., Dow Corning 9040 and 9011, Shin Etsu KSG 30, KSG 21, KSG 210, KSG 15AP), and silicone gums (e.g., Dow Corning 1502, Shin Etsu MK 15H). Polydiorganosiloxane-polyoxyalkylene copolymers (dimethicone polyols), are well known in the cosmetics and personal care arts and are described, for example, in U.S. Pat. No. 4,265,878, incorporated herein by reference, which further describes the copolymers and their methods of preparation. Briefly, polydiorganosiloxane-polyoxyalkylene copolymers are characterized by at least one polydiorganosiloxane segment and at least one polyoxyalkylene segment. The polydiorganosiloxane segment consists essentially of RₙSiO₍₄₋ₙ₎ units wherein n has a value of from 0 to 3 inclusive. There is an average of approximately two R radicals per siloxane unit in the copolymer with each R denoting a radical selected from the group consisting of methyl, ethyl, vinyl, phenyl and a divalent radical bonding the polyoxyalkylene segment to the polydiorganosiloxane segment. Useful polyoxyalkylene segments have an average molecular weight of from about 1000 to about 5000 and consist of from 0 to about 60 mole % polyoxypropylene units and from about 40 to about 100 mole % polyoxyethylene units. Polyoxyalkylene segments consisting of from about 40 to about 60 mole % of polyoxypropylene units and from about 40 to about 60 mole % of polyoxyethylene units are more preferred with segments consisting of an equimolar mixture of polyoxypropylene and polyoxyethylene units being most preferred. A terminal portion of each polyoxyalkylene segment is bonded to the polydiorganosiloxane segment.

The crosslinked siloxane elastomers are also well known in the cosmetic formulational arts, and are disclosed, for example, in U.S. Pat. No. 5,942,215, incorporated herein by reference. Siloxane elastomers are crosslinked or partially crosslinked, entangled, viscoelastic polymer networks, preferably made by the platinum catalyzed reaction known as hydrosilation of vinyl silicone fluids by either hydrosiloxane fluids or highly branched MQ hydride fluids. Control of the stoichiometry and type of the vinyl silicone fluid and the silanic crosslinker controls the properties of the cured networks. Additional vinyl reactants, such as vinyl alkenes can be introduced in the reactive medium to further modify the silicone network. The choice of the reaction solvent is also a means to modify the properties of the resultant materials. The average molecular weight of the silicone elastomers is between about 10,000 and about 40 million, and is preferably between about 10,000 and about 20 million. Typically, the crosslinked siloxane polymeric networks are swollen substantially by oily materials, preferably silicone fluids such as cyclomethicone and/or dimethicone.

Preferred crosslinked silicone materials are described in U.S. Pat. No. 5,654,362, which is incorporated herein by reference. An example of such a material is Dow 9040, commercially available from Dow Corning Corporation, Midland, Mich.

High molecular weight silicone carriers contemplated for use in the present composition also include organosiloxane polymers optionally dispersed in cyclopentasiloxane, such as cyclopentasiloxane and dimethicone/vinyl trimethylsiloxy silicate crosspolymers sold under the tradename “Si-Tec RE 100” (available from ISP Technologies, Wayne, N.J.). This material is a non-emulsfying organosiloxane polymer dispersed in cyclopentasiloxane, and takes the form of a semi-solid, creamy and semi-transparent resin gel. Si-Tec RE-100 delivers a substantive hydrophobic film to the skin, which-increases water resistance and improves the adhesion of a variety of ingredients including powders, pigments and actives.

Other carriers known in the art may also be implemented in the composition of the invention, for example shea butter, or known equivalents such as cocoa butter and the like; hydrogenated castor oil; capric/caprylic triglyceride; ceresine wax, or equivalents, such as ozokerite, micro crystalline wax, and the like; petrolatum; cetyl dimethicone copolyol; BIS-diglyceryl polyacyladipate-2; behenoyl stearic acid; dibehenoyl fumarate; polyethylene glycol-30 dipolyhydroxystearate; and combinations thereof.

Preferably, the amount of high molecular weight silicone carrier in the composition of the invention ranges from about 25 to about 50 wt %, more preferably from about 30 to about 45 wt %, and most preferably from about 33 to about 43 wt %, all percentages based on the total weight of the composition.

As indicated above, the composition of the invention is substantially free of water. Absence of water in the composition of the present invention prevents crystals of hydroquinone from forming and giving the composition a grainy feel. In addition, the absence of water in the composition maintains the integrity and reduces the rate of oxidation of the hydroquinone for a longer time, relative to aqueous hydroquinone compositions. This effect significantly increases the shelf-life of the product and eliminates the need for reducing agents, such as sulfites, which are malodorous and can irritate the skin. As defined herein, the phrase “substantially free of water” refers to amounts of water of approximately 0.1% by weight or less based on the total weight of the composition.

Additional ingredients or additives may also be added to the composition of the present invention, and include salicylic acid, vitamins, sunscreens, preservatives, essential oils, fragrances, colorants, firming agents, anti-wrinkle agents, free-radical scavengers, chelating agents, moisturizers, humectants, silicone elastomer powders, such as Dow Corning 9506 powder, as well as combinations of these. In general, these additional additives preferably comprise from about 0.1 to about 5% by weight, more preferably from about 0.1 to about 1.5% by weight, based on the total weight of the composition.

In general terms, the composition of the present invention is made by first combining the desired amounts of hydroquinone and anhydrous solvent. During this step, the mixture is stirred at temperatures ranging from room temperature (20° C.) to about 70° C. until all the solid crystals of hydroquinone are solubilized and the mixture is homogeneous. Elevated temperatures generally expedite solubilization of the hydroquinone to form an anhydrous solvent containing dissolved hydroquinone, and are therefore preferred. Mixing times generally range from about 3 minutes to 24 hours or more, depending on the processing temperature. It is within the skill of one in the art to determine the appropriate mixing time and temperature to achieve homogeneous mixing results.

Following dissolution of the hydroquinone, additional additives are (if desired) and high molecular weight silicone carrier are separately combined and added to the mixture containing the dissolved hydroquinone. This separate mixing step takes place under the same conditions as the above mixing step. These two combinations are then added together and mixed until a homogeneous mixture is formed. Mixing times during this step generally range from 3 minutes to 24 hours or more at temperatures ranging from about room temperature (20° C.) to about 70° C. Alternatively, it is possible to add the high molecular weight silicone carrier and additional additives individually and mix as described above.

During use, the skin care composition of the present invention may be applied to small, individual skin areas (dark spots, for example), or larger areas, such as the face or arms. The amount of the composition used will depend on how much skin area need be covered. In one embodiment, ranges of useful amounts of the composition of the present invention range from about 0.1 grams (for spots) to about 2 grams (for an entire arm or face).

The following Examples are intended to illustrate, but in no way limit the scope of the present invention. All parts and percentages are by weight and all temperatures are in degrees Celsius unless explicitly stated otherwise.

### EXAMPLE 1

Preparation of an Anhydrous Topical Skin Care Composition Containing 2% Hydroquinone

The following ingredients (Phase A) were combined using a Lightnin mixer (Rochester, N.Y.):

| Ingredient | Amount (wt %) |
|---|---|
| Butylene Glycol | 6.0 |
| Hydroquinone | 2.0 |
| Willow Herb extract in Glycerin | 1.0 |

These ingredients were mixed for approximately 30 minutes at 60° C. Following preparation of Phase A, Phase B was prepared separately by combining the following ingredients:

| Ingredient | Amount (wt %) |
|---|---|
| Salicylic Acid | 1.0 |
| Dimethicone | 1.0 |
| Cyclomethicone | 7.0 |
| Tocopheryl Acetate | 0.5 |
| Retinyl Palmitate | 0.25 |
| Dow Corning 9506 | 1.5 |
| (silicone elastomer powder) | |
| Panthenol (alcohol) | 0.25 |
| Cross-linked Silicone Elastomer | 40.0 |
| (Dow Silicone 9040) | |
| Cyclopentasiloxane and Dimethicone/Vinyl | 39.4 |
| Trimethylsiloxy Silicate Crosspolymers | |
| (Si Tech Re 100) | |
| Lavender Oil | 0.1 |

Like Phase A, Phase B was prepared by mixing the ingredients in a Lightnin mixer for approximately 30 minutes at 60° C. Finally, Phase A was added to Phase B, and mixed in the Lightnin mixer for 30 minutes at 60° C.

Between 0.1 and 2 grams of the above composition were applied on a daily basis to the skin of an individual having skin spots. After two weeks, the skin spots were noticeably reduced.

### EXAMPLE 2

Preparation of an Anhydrous Topical Skin Care Composition Containing 3% Hydroquinone

The following ingredients (Phase A) were combined using a Lightnin mixer (Rochester, N.Y.):

| Ingredient | Amount (wt %) |
|---|---|
| Propylene Glycol | 8.0 |
| Hydroquinone | 3.0 |
| Willowherb Extract in Glycerin | 1.0 |

These ingredients were mixed for approximately 30 minutes at 60° C. Following preparation of Phase A, Phase B was prepared separately by combining the following ingredients:

| Ingredient | Amount (wt %) |
|---|---|
| Salicylic Acid | 0.5 |
| Phenyltrimethicone | 1.0 |
| Cyelomethicone | 5.0 |
| Tocopheryl Acetate | 0.5 |
| Retinyl Palmitate | 0.25 |
| Gransil EPLS | 1.5 |
| Panthenol | 0.25 |
| Cross-Linked Silicone Elastomer | 38.0 |
| (Dow Silicone 9040) | |
| Cyclomethicone and Dimethicone | 40.9 |
| Crosspolymer-3 | |
| Ginger Oil | 0.1 |

Like Phase A, Phase B was prepared by mixing the ingredients in a Lightnin mixer for approximately 30 minutes at 60° C. Finally, Phase A was added to Phase B, and mixed in the Lightnin mixer for 30 minutes at 60° C.

Between 0.1 and 2 grams of the above composition were applied on a daily basis to the skin of an individual having skin spots. After two weeks, the skin spots were noticeably reduced.

### EXAMPLE 3

Preparation of an Anhydrous Topical Skin Care Composition Containing 4% Hydroquinone

The following ingredients (Phase A) were combined using a Lightnin mixer (Rochester, N.Y.):

| Ingredient | Amount (wt %) |
|---|---|
| Butylene Glycol | 10.0 |
| Hydroquinone | 4.0 |
| Willowherb Extract in Glycerin | 1.0 |

These ingredients were mixed for approximately 30 minutes at 60° C. Following preparation of Phase A, Phase B was prepared separately by combining the following ingredients:

| Ingredient | Amount (wt %) |
|---|---|
| Salicylic Acid | 0.75 |
| C₂₀₋₂₄ Alkyldimethicone | 1.0 |
| Cyelomethicone | 5.0 |
| Tocopheryl Acetate | 0.75 |
| Retinyl Palmitate | 0.25 |
| Gransil SRP | 2.0 |
| Panthenol | 0.25 |
| Si Tech Re 100 | 36.0 |
| Cyclomethicone and Dimethicone | 38.9 |
| Crosspolymer-3 | |
| Roman Chamomile Oil | 0.1 |

Like Phase A, Phase B was prepared by mixing the ingredients in a Lightnin mixer for approximately 30 minutes at 60° C. Finally, Phase A was added to Phase B, and mixed in the Lightnin mixer for 30 minutes at 60° C.

Between 0.1 and 2 grams of the above composition were applied on a daily basis to the skin of an individual having skin spots. After two weeks, the skin spots were noticeably reduced.

While the invention has been described above with reference to specific embodiments thereof, it is apparent that many changes, modifications, and variations can be made without departing from the inventive concept disclosed herein. Accordingly, it is intended to embrace all such changes, modifications, and variations that fall within the spirit and broad scope of the appended claims. All patent applications, patents, and other publications cited herein are incorporated by reference in their entireties.

What is claimed is:

1. An anhydrous topical skin care composition, comprising:
(a) hydroquinone;
(b) an anhydrous solvent; and
(c) a high molecular weight silicone carrier;
wherein said composition is substantially free of water.

2. The anhydrous topical skin care composition of claim 1, wherein said hydroquinone comprises from about 0.5 to about 10% by weight, based on the total weight of said composition.

3. The anhydrous topical skin care composition of claim 1, wherein said hydroquinone comprises from about 1 to about 7% by weight, based on the total weight of said composition.

4. The anhydrous topical skin care composition of claim 1, wherein said hydroquinone comprises from about 2 to about 4% by weight, based on the total weight of said composition.

5. The anhydrous topical skin care composition of claim 1, wherein said anhydrous solvent is selected from the group consisting of monohydric alcohols, dihydric alcohols (glycols), trihydric alcohols (glycerols), and combinations thereof.

6. The anhydrous topical skin care composition of claim 5, wherein said anhydrous solvent comprises one or more dihydric alcohols (glycols) selected from the group consisting of butylene glycol, propylene glycol, pentylene glycol, ethylene glycol, polyethylene glycol, hexylene glycol, ethoxydiglycol, methoxypolyethylene glycol, and combinations thereof.

7. The anhydrous topical skin care composition of claim 5, wherein said anhydrous solvent comprises glycerol or dimethyl isosorbide as the trihydric alcohol.

8. The anhydrous topical skin care composition of claim 1, wherein said anhydrous solvent comprises from about 1 to about 20% by weight, based on the total weight of said composition.

9. The anhydrous topical skin care composition of claim 1, wherein said anhydrous solvent comprises from about 4 to about 14% by weight, based on the total weight of said composition.

10. The anhydrous topical skin care composition of claim 1, wherein said anhydrous solvent comprises from about 4 to about 8% by weight, based on the total weight of said composition.

11. The anhydrous topical skin care composition of claim 1, wherein said a high molecular weight silicone carrier is a polydiorganosiloxane-polyoxyalkylene copolymer (dimethicone copolyol).

12. The anhydrous topical skin care composition of claim 1, wherein said high molecular weight silicone carrier is a cross-linked silicone elastomer.

13. The anhydrous topical skin care composition of claim 1, wherein said high molecular weight silicone carrier is a silicone gum.

14. The anhydrous topical skin care composition of claim 1, wherein said high molecular weight silicone carrier is an organosiloxane polymer optionally dispersed in cyclopentasiloxane.

15. The anhydrous topical skin care composition of claim 1, wherein said high molecular weight silicone carrier is a cyclopentasiloxane and dimethicone/vinyl trimethylsiloxy silicate crosspolymer.

16. The anhydrous topical skin care composition of claim 1, wherein said high molecular weight silicone carrier comprises from about 25 to about 50% by weight, based on the total weight of said composition.

17. The anhydrous topical skin care composition of claim 1, wherein said high molecular weight silicone carrier comprises from about 30 to about 45% by weight, based on the total weight of said composition.

18. The anhydrous topical skin care composition of claim 1, wherein said high molecular weight silicone carrier comprises from about 33 to about 43% by weight, based on the total weight of said composition.

19. The anhydrous topical skin care composition of claim 1, further comprising additional additives selected from the group consisting of salicylic acid, vitamins, sunscreens, preservatives, essential oils, fragrances, colorants, firming agents, anti-wrinkle agents, free-radical scavengers, chelating agents, moisturizers, humectants, silicone elastomer powders, and combinations thereof.

20. The anhydrous topical skin care composition of claim 19, wherein said additional additives comprise from about 0.1 to about 5% by weight, based on the total weight of said composition.

21. The anhydrous topical skin care composition of claim 19, wherein said additional additives comprise from about 0.1 to about 1.5% by weight, based on the total weight of said composition.

22. A method of making an anhydrous topical skin care composition, comprising the steps of:
(a) combining hydroquinone and an anhydrous solvent to form an anhydrous solvent containing dissolved hydroquinone;
(b) combining said anhydrous solvent containing dissolved hydroquinone with a high molecular weight silicone carrier to form said anhydrous topical skin care composition.
